(19) United States
(12) Patent Application Publication
(10) Pub. No.: US 2004/0067890 A1
(43) Pub. Date: Apr. 8, 2004

(54) Ascorbic acid salts of organic bases with enhanced bioavailability for synergictic anti-aging and skin protective cosmetic compositions

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/265,000

(22) Filed: Oct. 4, 2002

(51) Int. Cl. A61K 38/05; A61K 31/737; A61K 31/7008; A61K 31/4433; A61K 31/375; C07K 5/06

(52) U.S. Cl. 514/18; 514/62; 514/474; 514/55; 514/336; 530/331; 536/18.7; 536/20; 546/284.4; 536/53

(57) ABSTRACT

This invention relates to in-situ preparation, and stable topical delivery systems of ascorbic acid salts of organic bases that provide skin beneficial properties, including reduction in signs of skin aging, anti-wrinkle, anti-oxidant, and photo-protection from UV and sunlight. The formulation avoids the use of oils, minimizes the importance of the pH of the formulation, allows the incorporation of an aqueous solution of ascorbic acid or alkali metal salts of ascorbic acid in the formulation, does not require packaging the formulation in air tight containers, allows the use of large amounts of ascorbic acid, its salts, and its derivatives, and does not require the use of expensive coatings. Moreover, several ascorbic acid derivatives of different chemical composition can be made in a stable topical formulation by the in-situ combination of readily available starting materials in a water solution, despite the understanding well known in the prior art that such compositions in water are inherently unstable. The in-situ method also permits the preparation of novel ascorbic acid derivatives that are not known in the prior art.

### BACKGROUND OF THE INVENTION

This invention relates to in-situ preparation, and application of ascorbic acid salts of organic bases that provide skin beneficial properties, including reduction in signs of skin aging, anti-wrinkle, anti-oxidant, and photo-protection from UV and sunlight. The in-situ method also permits the preparation of novel ascorbic acid derivatives.

A number of methods have been reported for enhancing the topical delivery of oil-soluble skin care ingredients. These methods have been discussed in U.S. Patent Application No. 20020022040 to Robinson et al. and U.S. Patent Application NO. 20020102280 to Anderson. However, the topical delivery of water-soluble skin, hair, and body care ingredients that are unstable in the presence of water and air have been difficult to prepare.

Topical compositions that contain ascorbic acid in any substantial amount are inherently unstable in the presence of water, air, and light. Various approaches are known for stabilizing ascorbic acid in various cosmetic, dermatological, and other compositions.

One such approach for slowing the degradation of ascorbic acid advocates placing ascorbic acid in low pH solutions. For example, one such composition incorporates ascorbic acid in an aqueous-alcoholic solution consisting of at least 80% water and having a pH less than 3.5. Another similar composition comprises an acidic aqueous ascorbic acid solution including an oxyethylenated sorbitan ester. This solution has a pH of 3.4. Still another similar composition comprises water in oil emulsion containing ascorbic acid. This emulsion has a pH of 3.5 or less. A disadvantage of these compositions is that a pH of 3.5 or less is well below the normal pH of the epithelium of an individual. Applying such a composition can irritate the skin.

Another way of stabilizing ascorbic acid is to use a coating technique.

A further stabilization technique is to granulate the ascorbic acid. This technique, along with the coating technique mentioned above, tends to be expensive and may cause degradation of the ascorbic acid. Further, ascorbic acid granules can be impractical when used in compositions that are applied to the skin.

Still another stabilization technique utilizes metal salts of phosphorylated ascorbic acid in a cosmetic composition. The pH of the composition is 8 to 9. This pH is significantly higher than the normal pH of skin.

Still a further stabilized composition comprises small crystals of Ascorbic acid in a carrier that is essentially water free and includes less than 0.5% water. Eliminating water from a composition is one commonly used approach for increasing the stability of ascorbic acid in the composition.

Yet another stabilized composition comprises ascorbic acid in the form of a fat-soluble fatty acid ester in a carrier.

Yet a further stabilized composition comprises ascorbic acid in a water in oil emulsion.

Yet still a further stabilized composition consists of particles dispersed in an oil or other non-aqueous phase. The particles include a water-soluble or water-dispersible polymer, ascorbic acid, water, and a surfactant. While oil tends shield ascorbic acid from oxygen and water, the oil, when applied to the skin, produces a feel that typically is not preferred by customers.

The foregoing approaches for stabilizing ascorbic acid are set forth in U.S. Pat. No. 6,103,267 to Mitchnick et al.

A further approach for stabilizing ascorbic acid is to seal an aqueous solution of ascorbic acid tightly in a container to prevent air from reaching the solution. See U.S. Pat. No. 6,238,713.

Another approach for stabilizing ascorbic acid is to incorporate only small amounts, typically less than 1%, of ascorbic acid into a composition. See, for example, U.S. Pat. No. 6,136,330.

It would be highly desirable to provide a stabilized formulation of ascorbic acid and its water-soluble derivatives would not have an unusually low pH or an unusually high pH, that would not require granules of ascorbic acid, that would not require expensive coating techniques, that would not require the formulation be sealed in an air tight container, that would not require that only small amounts of ascorbic acid be utilized, and that would not require the use of an oil or other hydrophobic composition. In addition, it would be highly desirable if skin, hair, and body beneficial properties of ascorbic acid can be further enhanced to provide increased bioavailability of ascorbic acid, and synergistic skin, hair, and body beneficial properties in combination with another skin, hair, and body beneficial ingredient, such as an organic base, provided that such as organic base form a salt with ascorbic acid, and such a salt of ascorbic acid with an organic base provide skin, hair, and body beneficial properties in synergism with each other.

### OBJECTS OF THE INVENTION

This invention relates to in-situ preparation and application of ascorbic acid salts of organic bases that provide skin beneficial properties, including reduction in signs of skin aging, anti-wrinkle, anti-oxidant, and photo-protection from UV and sunlight.

This invention also relates to compositions that include ascorbic acid salts of organic bases that are prepared in-situ from ascorbic acid and organic bases, or their derivatives.

More particularly, the invention relates to ascorbic acid salts of organic bases that provide enhanced stability of ascorbic acid and enhanced bioavailability of both ascorbic acid and organic base moieties.

In a further respect, the invention relates to ascorbic acid salts of organic bases that stabilize ascorbic acid part of composition that does not require the compositions have a low pH.

In another respect, the invention relates to ascorbic acid salts of organic bases that do not rely on a non-aqueous organic to stabilize the ascorbic acid part of composition from oxygen, light, or water.

In yet another respect, the invention relates to ascorbic acid salts of organic bases that utilize water to stabilize ascorbic acid part of composition.

In a yet another respect, the invention relates to in-situ preparation ascorbic acid salts of organic bases from commonly available ascorbic acid and organic bases, or their derivatives.

In a yet another respect, the invention relates to in-situ preparation of ascorbic acid derivatives that are new and not known in the prior art.

### BRIEF DESCRIPTION OF THE INVENTION

I have discovered an improved stabilized formulation of ascorbic acid derivatives and an in-situ method for making the same. The formulation avoids the use of oils, minimizes the importance of the pH of the formulation, allows the incorporation of an aqueous solution of ascorbic acid or various metal salts of ascorbic acid in the formulation, does not require packaging the formulation in air tight containers, allows the use of large amounts of ascorbic acid and its derivatives, and does not require the use of expensive coatings. Moreover, several ascorbic acid derivatives of different chemical composition can be made in a stable topical formulation by the in-situ combination of readily available starting materials in a water solution, despite the understanding well known in the prior art that such compositions in water are inherently unstable. The in-situ method also permits the preparation of novel ascorbic acid derivatives.

### DETAILED DESCRIPTION OF THE INVENTION

The stabilized ascorbic acid topical formulation of the current invention comprises ascorbic acid or metal salts of ascorbic acid; water; an organic base or an acid salt of an organic base, that can chemically bind with ascorbic acid molecule to form in-situ an ascorbic acid salt of an organic base; and a miscible organic solvent. The formulation is particularly suited for, but not limited to, use in cosmetic and medical fields as a composition to be applied externally to the skin and hair of an individual. Additional skin beneficial and cosmetically desirable ingredients can be added to the formulation.

As is known in the art, the union of an acid and base leads to the formation of a salt as part of a neutralization reaction. In the case of diacid and triacid bases, and of dibasic and tribasic acids, the mutual neutralization may vary in degree, producing respectively basic, neutral, or acid salts. A method for synthesizing water-soluble, single component, or multi-component salts of ascorbic acid has now been discovered, which includes reacting ascorbic acid in water with at least one organic base to form a single component salt, or several organic bases to form a multi-component salt, the quantity of organic base or bases depending upon the molecular weight and acidity of organic base or bases to form salts with ascorbic acid. While the preparation of such salts is not difficult, as set forth in U.S. Patent Application No. 20020058704 to Malik et al., the preparation of such salts of ascorbic acid, in water solution, that are stable in the presence of water and air, has been difficult in prior art.

The amount of salt or a mixture of salts of ascorbic acid with one or a mixture of organic bases in the formulation is 0.1% to 40% by weight, preferably 5% to 20% by weight. A particular advantage of the ascorbic acid organic base salt stabilized formulation of the invention is that relatively large amounts of ascorbic acid or its salts, up to about 40% by weight, can be incorporated in the formulation. If the amount of ascorbic acid salts is in excess of 40%, the amount of water required becomes excessive. Consequently, quantities of ascorbic acid salts greater than about 40% are presently not practical in the practice of the invention. It is theorized that this is the case because as more ascorbic acid salt is utilized, more water is required. And, the amount of water appears limited by the amount of water-miscible organic solvent required.

The amount of the water miscible organic solvent in the formulation is 1% to 80%, preferably 10% to 50% by weight. The use of a water miscible organic solvent is believed critical in the practice of the invention because it facilitates the intermixing of water—which contains ascorbic acid salts-with the organic solvent. Further, it is preferred that the organic solvent be water absorbent and absorb at least a portion of the water and ascorbic acid salts in the formulation. The water absorption characteristic of the organic solvent is, as is the case with the surfactant, important in the formulation of the invention because when water is bound with a hydrophilic or water-absorbing ingredient, the pH of the formulation ordinarily is not that important with respect to the stability of the ascorbic acid salts. The water absorption characteristic is also important because it facilitates the water miscible characteristic of the organic solvent. The amount of water in the formulation is from 1% to 60%, preferably 5% to 25%. As a general rule, excluding water from ascorbic acid salts increases the stability of the ascorbic acid salts. Water facilitates the oxidation and instability of ascorbic acid salts. However, in the invention water is desirable and required. Surprisingly, water helps stabilize the formulation. It is theorized, although not proven, that the water solubilizes the ascorbic acid salts and thereby facilitates the mixing of these salts in the water miscible organic solvent.

The proportion of water to water-miscible organic solvent in the formulation appears to be important in terms of stability of ascorbic acid salts, and is in the range of 1:2 to 1:6, preferably 1:3 to 1:5. This proportion is believed important (although this belief has not presently been proven) because there must be sufficient water-miscible organic solvent to absorb and/or mix with water containing ascorbic acid salts to stabilize such salts.

If a surfactant is desired, then the amount of surfactant in the formulation is 1% to 30%, preferably 20% to 30% by weight. It is possible that the amount of surfactant in the formulation can be up to 40% by weight, but concentrations of surfactant greater than 30% increase the risk that the surfactant may gel out. Less than 20% by weight of surfactant is acceptable, but the foaming properties of the formulation are not as good. The surfactant is preferably hydrophilic, and absorbs a portion of the water and vitamin C in the formulation. The water absorption characteristic of the surfactant is important in the practice of the invention because when water is bound with a hydrophilic or water-absorbing ingredient, the pH of the formulation ordinarily is not that important with respect to the stability of the vitamin C.

To illustrate the scope of this invention, the equation 1 shows the formation of an ascorbic acid salt of an organic base in water solution;

Ascorbic Acid+Organic Base [symbol] Ascorbic Acid salt of Organic Base (Equation 1)

Thus, by mixing ascorbic acid with glucosamine in equimolar amounts in water solution, one mole of Glucosamine Ascorbate is produced in-situ, as illustrated in Equation 2.

Ascorbic Acid+Glucosamine [symbol] Glucosamine Ascorbate (Equation 2)

Additionally, by mixing a metal salt of ascorbic acid with an acid salt of an organic base, ascorbic acid salt of an organic base can be prepared in-situ, as depicted in Equation 3.

Sodium Ascorbate+Glucosamine Hydrochloride [symbol] Glucosamine Ascorbate+Sodium Chloride (Equation 3).

Multi-component ascorbic acid compositions can thus be made in-situ by mixing the reacting components in proportionate molar quantities in water solution, as illustrated in Equation 4.

Ascorbic Acid+Glucosamine+Allantoin+Carnosine+Dimethylaminoethanol [symbol] Glucosamine Ascorbate+Allantoin Ascorbate+Carnosine Ascorbate+Dimethylaminoethanol Ascorbate (Equation 4)

New, previously unknown ascorbic acid derivatives can be made by in-situ method of present invention, as illustrated for the preparation of Glucosamine Ascorbyl Phosphate in Equation 5.

Sodium Ascorbyl Phosphate+Glucosamine Hydrochloride [symbol] Glucosamine Ascorbyl Phosphate+Sodium Chloride (Equation 5)

Multi-component compositions of both previously unknown ascorbic acid derivatives and previously known ascorbic acid derivatives can also be made, as illustrated in Equation 6.

Sodium Ascorbyl Phosphate+Glucosamine Hydrochloride+Arginine Hydrochloride+Niacinamide Hydrochloride+Lidocaine Hydrochloride [symbol] Glucosamine Ascorbyl Phosphate+Arginine Ascorbyl Phosphate+Niacinamide Ascorbyl Phosphate+Lidocaine Ascorbyl Phosphate+Sodium Chloride (Equation 6)

The examples shown in Equation 1 to Equation 6 are only illustrative, and they do not represent any limitations or scope of present invention.

In prior art, the preparation of such salts of ascorbic acid with organic bases has been very difficult, inconvenient, or even dangerous.

For example, Niacinamide Ascorbate, a salt formed by the reaction of niacinamide (an organic base) and ascorbic acid, has been possible only with special handling, as reported by C. W. Bailey et al., J Amer. Chem. Soc., 67, 1184-5, (1945). Similarly, Chitosan Niacinamide Ascorbate salt has been reported in U.S. Patent Application No. 20020058704 in a water solution from niacinamide and ascorbic acid, but this involves a two-step process whereby niacinamide ascorbate must first be prepared and then reacted with chitosan in a second chemical step. Moreover, the stability of chitosan niacinamide ascorbate in contact with water, or any synergistic skin, hair, or body beneficial properties have not been reported. It would thus be advantageous to prepare chitosan niacinamide ascorbate in one single chemical step from readily available ingredients for its direct formulation in skin beneficial cosmetic preparations.

As another example, glucosamine ascorbate, which is a salt of ascorbic acid with glucosamine, an organic base, has been used in certain cosmetic preparations, U.S. Pat. No. 6,358,539 to Murad. However, its preparation from readily available ingredients, or its stability in formulations that contain substantial amounts of water has not been reported. Ascorbyl glucosamine, probably the same ingredient as glucosamine ascorbate, has been used for wound healing compositions, but only in combination with other pharmaceutical ingredients, as set forth in U.S. Patent Application No. 20010006646 to Coine. Chondroitin and Glucosamine derivatives have been used in combination with ascorbic acid in solid oral dosage forms for enhanced relief of joint disorders, as set forth in U.S. Pat. No. 6,162,787 to Sorgente et al. However, the preparation of stable salts of ascorbic acid with chondroitin or glucosamine, or the topical application of such salts were not reported by Sorgente et al.

As an additional example, a mixture of ascorbic acid with arginine, an organic base, has been made by physical mixing of the two ingredients in a powder form by physical mixing of arginine powder with ascorbic acid powder. Said mixture prevents the browning of ascorbic acid after long-term storage. However, stability of such a mixture in the presence of water is not known. It is also not known if arginine and ascorbic acid have reacted to form the corresponding salt, arginine ascorbate, in the solid state mixing process. The mixture prevents peroxidative injuries of cells, when taken orally. Any skin beneficial effects from its topical application are not known. The above have been set forth in U.S. Patent Application No. 20020091156 to Kimoto et al.

As a further example, ascorbic acid is reported to enhance the bioavailability of lysine, an organic base, when administered orally, for HIV treatment. Additionally, ascorbate provides an antioxidant defense against the herpes virus, which tends to lose latency during period of oxidative, free radical excess. These are set forth in U.S. Patent Application No. 20010031737 to Richardson et al. A convenient preparation of stable lysine ascorbate and its beneficial applications in topical skin, hair, and body care compositions are of considerable importance. While arginine and lysine have been combined with ascorbic acid, as illustrated above, for beneficial compositions, the small peptides of lysine and arginine, such as lysyl-arginine, or arginyl-lysine have not been combined with ascorbic acid, although such dipeptides have been shown to possess beneficial properties, as set forth in U.S. Pat. No. 5,811,401 to Bucala et al.

As a yet further example, glutathione, an organic base, has been stabilized by ascorbic acid by combining them in a solid state in an oral dosage form. The formation of any glutathione ascorbate salt in these mixtures is not established, although such mixtures have been shown to impart physiologically beneficial properties, as set forth in U.S. Pat. No. 6,423,687 to Demopolos et al.

As a yet another example, creatine ascorbate has been reported in U.S. Pat. No. 6,242,491, to Kaddurah as energy generating systems and antioxidants for preservation of skin against adverse aging effects and damage secondary to insults such as harmful sun radiations, stress and fatigue, when given orally. Pischel et al., U.S. Pat. No. 5,863,939, claim that creatine ascorbate can be used for enhancing muscular development and strength in the field of sport, as prophylactics against and for the treatment of conditions of oxygen deficit (ischemia) and as immune system stimulants in the field of health, for the treatment of muscular atrophy and as food supplements. However, the preparation of creatine ascorbate reported by Pischel et al. requires a solvent selected from the group consisting of an alcohol, an ether, a ketone, an, ester, an aliphatic hydrocarbon and an aromatic hydrocarbon. This causes serious problems in the removal of trace quantities of such solvents form the product, as some of such solvents such as aromatic hydrocarbons are known to be toxic and cancer causing. The in-situ preparation of creatine ascorbate in water as a solvent would be far more convenient and safe.

The role of glutathione, as glutathione ascorbate, in Alzheimer's disease, and other pharmaceutical applications, has been well recognized, as set forth in U.S. Pat. Nos. 6,423,687 and 6,350,467; both to Demopolos. A convenient in-situ preparation of Glutathione Ascorbate should be of significant contribution to this field of medical research.

In the above examples, organic bases such as glucosamine, niacinamide, chitosan, arginine, lysine, chondroitin, creatine, and glutathione, etc. have been used in combination with ascorbic acid. These organic bases themselves have skin, hair, and body beneficial properties upon their topical application. The combination of such organic bases with ascorbic acid provides yet additional, synergistic benefits. It would thus be advantageous to devise a simple method for the preparation of stable ascorbic acid salts of such skin, hair, and body beneficial organic bases that can be utilized in cosmetic preparations that may contain water in substantial amounts without any special exclusion of air or light. Moreover, it would be additionally advantageous if such salts can be made in-situ, thus not requiring their preparation in a separate step. It would be additionally advantageous if such salts can be made from readily available ingredients.

In addition, it is well known that mixtures of several ascorbic acid salts are more beneficial than the use of single components alone. For example, U.S. Pat. No. 6,110,966 to Pollock describes a triple action complex containing three forms of vitamin C, which provides the triple action of a major anti-oxidant, an anti-inflammatory and a collagen booster. The triple action complex contains ascorbic acid, sodium ascorbate, and ascorbyl glucosamine as the three forms of vitamin C. U.S. Pat. No. 5,626,883 to Paul, describes a vitamin C supplement comprising ascorbic acid, ascorbyl palmitate, niacinamide ascorbate, calcium ascorbate, magnesium ascorbate, potassium ascorbate, and sodium ascorbate which together can be administered to a human to avoid the transitory initial suppression of human NK cell activity which is present when ascorbic acid alone is administered. U.S. Pat. No. 5,895,652 to Giampapa describes an ascorbate-citrus antioxidant complex, including: Vitamin C (from calcium, magnesium and niacinamide ascorbate), Vitamin C (ascorbic acid), ascorbyl palmitate (preferably fat soluble), and acerola juice powder (a natural form of Vitamin C) mixture for enhanced benefits. In these examples, various forms of ascorbic acid are individually prepared, and then mixed in correct proportions. It would be advantageous if such preparations can be made in-situ by mixing readily available ingredients, as that would avoid any errors that may be encountered in multiple mixing of ingredients, and it would offer manufacturing ease in the preparation of cosmetic or pharmaceutical products for topical application.

The formulations of present invention can also contain additional skin, hair, and body beneficial ingredients, such as surfactants, rheology modifiers, perfumes, antioxidants, preservatives, other anti-aging ingredients, vitamins, hormones, analgesics, anesthetics, sun screens, skin whiteners, anti-acne agents, anti-bacterial agents, anti-fungal agents, botanical extracts, pharmaceuticals, processing-aids, and such.

Examples of surfactants that can be utilized are anionic, amphoteric, nonionic and cationic surfactants. Examples of anionic surfactants include, without limitation, soaps, alkyl sulfates, anionic acyl sarcosinates, methyl acyl taurates, N-acyl glutamates, acyl isethionates, alkyl phosphate esters, ethoxylated alkyl phosphate esters, alkyl sulfosuccinates, trideceth sulfates, protein condensates, mixtures of ethoxylated alkyl sulfates, and the like. Examples of anionic non-soap surfactants are, without limitation, the alkali metal salts of organic sulfate having in their molecular structure an alkyl radical containing from about 8 to about 22 carbon atoms and a sulfonic acid or sulfuric acid ester radical. Examples of Zwitterionic surfactants are, without limitation, derivatives of aliphatic quaternary ammonium, phosphonium, and sulfonium compounds, in which the aliphatic radicals can be straight chain or branched and wherein one of the aliphatic substituents contains from about 8 to 18 carbon atoms and one contains an anionic water-solubilizing group, e.g., carboxyl, sulfonate, sulfate, phosphate, or phosphonate. Examples of amphoteric surfactants are, without limitation, derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxyl, sulfonate, sulfate, phosphate, or phosphonate. Examples of cationic surfactants are, without limitation, stearyldimenthylbenzyl ammonium chloride; dodecyltrimethyl ammonium chloride; nonylbenzylethyldimethyl ammonium nitrate; and tetradecylpyridinium bromide. Nonionic surfactants include, without limitation, compounds produced by the condensation of alkylene oxide groups (hydrophilic in nature) with an organic hydrophobic compound, which may be aliphatic or alkyl aromatic in nature, for example, the polyethylene oxide condensates of alkyl phenols.

Examples of rheology modifiers include, without limitation, various Carbomers, Carbopols (Acrylates/C10-30 Alkyl Acrylate Crosspolymer), Xanthan gum, Gellan Gum, Gum Arabic, Bentonite, various Clays, Silicas, Fumed Silica, Zeolites, Structure Plus (Acrylates/Aminoacrylates/C10-30 Alkyl PEG-20 Itaconate Copolymer), Structure XL, Aristoflex AVC (Ammonium Acryloyldimethyltaurate), and such.

The following terms used herein have the meanings set forth below.

Acidity of Organic Base. The combining power of an organic base with reference to an acid.

Amino Acid. Any of a group of organic compounds containing the amino group combined with the carboxyl radical.

Base. A compound that is capable of so uniting with an acid as to neutralize it and form a salt.

Basic. A compound that has base-like properties.

Derivative. A compound formed or regarded as being formed from a specified substance or another compound, usually by partial substitution.

Dispersion. An emulsion or suspension. Comprise the dispersed substance and the medium it is dispersed in.

Emulsion. Intimate mixture of two incompletely miscible liquids.

Equimolar. Of equivalent molecular weight.

Hydrophilic. Strong affinity for water.

Hydrophobic. Weak affinity for water.

Lipophilic. Strong affinity for fats or other lipids.

Miscible. Capable of mixing in any ratio without separation of the two phases. The mixture formed by a miscible liquid or solid is a solution.

Oleophilic. Strong affinity for oils.

Organic. Being, containing, or relating to carbon compounds, especially in which hydrogen is attached to carbon whether derived from living organisms or not.

Organic Base. An organic compound that also contains one or more nitrogen atoms that can bind with an organic or inorganic acid to form a salt.

Organic solvent. A solvent including a carbon compound. Examples include, without limitation, glycerin, PEG-6 (Polyethylene glycol 300), and Mpdiol glycol.

Salt. Any compound consisting of the cation of a base and the anion of an acid, combined in proportions that give a balance of electropositive and electronegative charges.

Signs of Skin Aging. These include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, skin lines, crevices, bumps, large pores (e.g., associated with adrenal structures such as sweat gland ducts, sebaceous glands, or hair follicles), or unevenness or roughness, loss of skin elasticity (loss and/or inactivation of functional skin elastin), sagging (including loss and/or damage to functional subcutaneous muscle tissue and including puffiness in the eye area and jowls), loss of skin firmness, loss of skin tightness, loss of skin recoil from deformation, discoloration (including under eye circles), blotching, shallowness, hyper pigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

Solution. A solid, liquid, or gas mixed homogeneously with a liquid.

Solvent. A substance capable of or used in dissolving or dispersing one or more other substances, especially a liquid component of a solution present in greater amount than the solute.

Suspension. Particles mixed in a fluid or a solid, but undissolved.

Synergism. The joint action of different substances in producing an effect greater than the sum of effects of all the substances acting separately.

Synergistic. Acting together

Water miscible organic solvent. An organic solvent that can be mixed with water in any ratio without separation of the water from the organic solvent. In the practice of the invention, the preferred (but not required) water miscible organic solvents are those commonly used in cosmetic applications, for example, glycerin, ethylene glycol, propylene glycol, butylene glycol, hexylene glycol, pyrrolidone, N-methyl pyrrolidone, dimethyl sulfoxide, dimethyl sulfone, polyethylene glycol, polypropylene glycol, methylpropanediol, and similar solvents.

### EXAMPLES

The following examples are presented by way of illustration, and not limitation, of the invention. In each example, the amount or proportion of each ingredient is in weight percent unless otherwise indicated. The color of the products, where indicated, was measured by Hunter Lab Color Sensor Model D25M. The color is measured quantitatively by this machine in L, a, and b scale mode, where L is for light/dark scale, a is for red/green scale, and b is for blue/yellow scale.

### Example 1

This example shows the in-situ preparation of niacinamide ascorbate and its in-situ use for the preparation of an example of a formulation prepared as per claim 9. Column 1 describes the ingredients as they are used in the formulation. Column 2 describes the final composition resulting from the in-situ formation of niacinamide ascorbate (an example of ascorbic acid—organic base complex as per claim 3).

| Ingredient | Column 1 | Column 2 |
|---|---|---|
| Glycerin (water miscible organic solvent) | 43.3 | 43.3 |
| Methyl paraben (preservative) | 0.2 | 0.2 |
| Ascorbic Acid | 9.0 | 0.0 |
| Niacinamide (nicotinamide) | 6.0 | 0.0 |
| Niacinamide Ascorbate | 0.0 | 15.0 |
| Deionized Water | 15.0 | 15.0 |
| Phenoxyethanol (preservative) | 0.9 | 0.9 |
| Tauranol I-78-6 (Sodium Cocoyl | 20.0 | 20.0 |
| Isethionate) (surfactant) | | |
| Tauranol ws conc. (Sodium Methyl | 5.0 | 5.0 |
| Cocoyl Taurate) (surfactant) | | |
| Actiplex 2789 (Extract of various plants) | 0.1 | 0.1 |
| Fragrance | 0.5 | 0.5 |

Procedure: All of the ingredients are mixed to form a composition. The mixture is heated and stirred at 60 to 70 degrees C. until the mixture is homogenous, i.e., for about five to ten minutes. The homogeneous mixture is cooled to room temperature. A paste-like product is formed. The stabilized niacinamide ascorbate formulation is used as a facial, hair, and body cleanser. It should be noted that when the composition is first mixed, as shown in Column 1, it is white in color. After preparation of the batch is complete, the product turns bright yellow, indicating the formation of niacinamide ascorbate, which is naturally yellow in color. The color meter readings were L 91.94, a −7.21, b 22.20.

### Example 2

The paste of Example I is stored at room temperature in a sealed container in the presence of air. After six months the paste is still yellow. A calorimetric reading with a color meter, such as Hunter Color Meter, shows that the color reading has changed by only 5%, and the product is still stable, and has not separated into solid and liquid phases. The color meter readings were L 91.43, a −7.03, b 24.46.

### Example 3

This example shows the in-situ preparation of an anti-aging, anti-wrinkle eye serum product that contains multi-component mixture of several ascorbic acid—organic base complexes, as per claim 14. Column 1 describes the ingredients as they are used in the formulation. Column 2 describes the final composition resulting from the in-situ formation of ascorbic acid—organic base complexes, per procedure described in this example.

| Ingredient | Column 1 | Column 2 | Column 3 |
|---|---|---|---|
| Deionized Water | 30.00 | 30.00 | 4.00 |
| Ascorbic Acid | 10.00 | 0.32 | 0.32 |
| Allantoin | 1.58 | 0.0 | 0.0 |
| Niacinamide | 1.22 | 0.0 | 0.0 |
| Lysine | 1.46 | 0.0 | 0.0 |
| Carnosine | 2.26 | 0.0 | 0.0 |
| Glutathione | 1.54 | 0.00 | 0.0 |
| Creatine | 1.31 | 0.00 | 0.0 |
| PEG-3 | 50.63 | 50.63 | 76.63 |
| Allantoin Ascorbate | 0.0 | 3.34 | 3.34 |
| Niacinamide Ascorbate | 0.0 | 2.98 | 2.98 |
| Lysine Ascorbate | 0.0 | 3.22 | 3.22 |
| Carnosine Ascorbate | 0.0 | 4.02 | 4.02 |
| Glutathione Ascorbate | 0.0 | 2.42 | 2.42 |
| Creatine Ascorbate | 0.0 | 3.07 | 3.07 |
| Total | 100.00 | 100.00 | 100.00 |

Procedure: All the ingredients in column 1 were mixed and heated at 60 to 70C for 30 minutes. A yellow mixture was obtained. The composition of this mixture is shown in Column 2. Water was then removed as much as possible in vacuo, and same amount of PEG-3 was added as the amount of water removed to give composition of product shown in Column 3. The product is Column 3 was obtained as a pale color syrup, useful for anti-age and anti-wrinkle eye zone applications.

### Example 4

### Face and Neck Gel

This example shows the in-situ preparation of an anti-aging, anti-wrinkle eye serum product that contains multi-component mixture of several ascorbic acid—organic base complexes, as per combination of claim 14 and 17. Column 1 describes the ingredients as they are used in the formulation. Column 2 describes the final composition resulting from the in-situ formation of ascorbic acid—organic base complexes, per procedure described in this example.

| Ingredient | Column 1 | Column 2 |
|---|---|---|
| Ascorbic Acid | 10.0 | 4.72 |
| Deionized Water | 20.0 | 20.0 |
| Geogard 221 (Preservative) | 0.5 | 0.5 |
| Glycerine Usp | 42.7 | 42.7 |
| Dow Corning 2501 Wax | 10.0 | 10.00 |
| Structure Plus | 4.0 | 4.00 |
| Eyebright Extract | 0.1 | 0.1 |
| Botanicals Extracts Blend | 0.1 | 0.1 |
| Vitamin E Acetate | 0.1 | 0.1 |
| Sodium Ascorbyl Phosphate | 3.23 | 0.00 |
| Arginine | 1.74 | 0.00 |
| Pyridoxine Hydrochloride | 2.05 | 0.00 |
| Carnosine | 2.26 | 0.00 |
| Glucosamine Hydrochloride | 2.05 | 0.00 |
| Allantoin | 1.58 | 0.00 |
| Pyridoxine Ascorbyl Phosphate | 0.00 | 2.35 |
| Arginine Ascorbate | 0.00 | 3.5 |
| Carnosine Ascorbate | 0.00 | 4.02 |
| Glucosamine Ascorbyl Phosphate | 0.00 | 2.40 |
| Allantoin Ascorbate | 0.00 | 3.34 |
| Sodium Chloride | 0.00 | 0.58 |
| Total | 100.00 | 100.00 |

Procedure: All the ingredients in column 1 were mixed and heated at 60 to 70C for 30 minutes. A yellow mixture was obtained. The composition of this mixture is shown in Column 2. The product is Column 2 was obtained as a pale color syrup, useful for face and neck zone anti-age and anti-wrinkle applications. The product has the following properties: pH 4.5; color meter readings L 44.50, a 0.19, b 40.86.

### Example 5

### Anti-Wrinkle Face Gel with In-Situ Preparation of Niacinamide Ascorbate

| Ingredients | Column 1 | Column 2 |
|---|---|---|
| PEG-6 | 46.5 | 46.5 |
| Ammonium Acryloylmethyltaurate | 1.0 | 1.0 |
| Glycerin Usp | 5.0 | 5.0 |
| Deionized Water | 20.0 | 20.0 |
| Geogard 221 (Preservative) | 0.5 | 0.5 |
| Vitamin E Acetate | 2.0 | 2.0 |
| Niacinamide | 6.0 | 0 |
| Ascorbic Acid | 9.0 | 0 |
| Dimethicone | 4.0 | 4.0 |
| Dimethiconol | 4.0 | 4.0 |
| Cetyl Dimethiconol | 2.0 | 2.0 |
| Niacinamide Ascorbate | 0.0 | 15.0 |

Procedure: All ingredients in Column 1 were mixed and heated at 40 to 50C for 30 minutes. The mixture was cooled to room temperature. A clear gel was obtained, with analysis reported in Column 2.

### Example 6

### The In-Situ Preparation of Glucosamine Ascorbate from Glucosamine and Ascorbic Acid as per Process in claim 14 is Shown in this Example

| Ingredients | Column 1 | Column 2 |
|---|---|---|
| Glucosamine | 17.9 | 0.0 |
| Ascorbic Acid | 17.6 | 0.0 |
| Deionized Water | 30.00 | 30.0 |
| Propylene Glycol | 34.5 | 34.5 |
| Glucosamine Ascorbate | 0.0 | 35.5 |

Procedure: All ingredients in Column 1 were mixed and heated at 40 to 50C for 30 minutes. The product was cooled. A thin solution of composition in Column 2 was obtained.

### Example 7

### The In-Situ Preparation of Glucosamine Ascorbate by a Process in claim 17 is Described in this Example

| Ingredients | Column 1 | Column 2 | Column 3 |
|---|---|---|---|
| Glucosamine Hydrochloride | 21.5 | 0.0 | 0.0 |
| Sodium Ascorbic | 19.8 | 0.0 | 0.0 |
| Deionized Water | 30.00 | 30.0 | 4.5 |
| Propylene Glycol | 28.7 | 28.7 | 59.5 |
| Glucosamine Ascorbate | 0.0 | 35.5 | 35.5 |
| Sodium Chloride | 0.0 | 5.8 | 0.5 |

Procedure: All ingredients in Column 1 were mixed and heated at 40 to 50C for 30 minutes. The product was cooled. A thin solution of composition in Column 2 was obtained. Water was evaporated in-vacuo, and same amount of propylene glycol was added as the amount of water removed. Upon cooling, sodium chloride precipitated, which was removed by filtration. The composition in Column 3 was thus obtained.

### Example 8

### The In-Situ Preparation of Glutathione Ascorbate is Described in this Example

| Ingredients | Column 1 | Column 2 |
|---|---|---|
| Glutathione | 30.7 | 0.0 |
| Ascorbic Acid | 17.6 | 0.0 |
| Deionized Water | 50.0 | 50.0 |
| Mehtylpropanediol | 1.7 | 1.7 |
| Glutathione Ascorbate | 0.0 | 48.3 |

Procedure: All ingredients in Column 1 were mixed. A solution of Glutathione Ascorbate was obtained, as shown in Column 2.

### Example 9

### Preparation of an Eye, face, and Neck Gel with Glucosamine Ascorbate, Niacinamide Ascorbate, and Other Skin Beneficial Ingredients

| Ingredient | Column 1 | Column 2 |
|---|---|---|
| PEG-6 | 54.28 | 54.28 |
| Aristoflex AVC | 1.00 | 1.00 |
| Glycerin USP | 5.00 | 5.00 |
| Deionized Water | 20.00 | 20.00 |
| Geogard 221 (preeservative) | 0.50 | 0.50 |
| Vitamin E Acetate | 0.50 | 0.5 |
| Niacinamide | 1.22 | 0.00 |
| Ascorbic Acid | 5.00 | 2.36 |
| Dimethicone | 4.00 | 4.00 |
| Dimethiconol | 4.00 | 4.00 |
| Cetyl Dimethiconol | 2.00 | 2.00 |
| Glucosamine Hydrochloride | 1.1 | 0.00 |
| Kaempferia Galanga Extract | 0.2 | 0.2 |
| Esculin | 0.5 | 0.5 |
| Boswellia Serrata Extract | 0.2 | 0.2 |
| Methylsulfonylmethane (MSM) | 0.5 | 0.5 |
| Niacinamide Ascorbate | 0.0 | 2.96 |
| Glucosamine Ascorbate | 0.0 | 1.8 |
| Sodium Chloride | 0.0 | qs |
| Total | 100.00 | 100.00 |

Procedure: Mix all ingredients in Column 1 and heat at 60 to 70C for 30 minutes. Cool to room temperature, and adjust pH to 4.5 with sodium hydroxide solution. A clear pale yellow gel of composition in Column 2 was obtained.

I claim:

1. An ascorbic acid salt of an organic base in a stabilized topical formulation comprising at least one salt of ascorbic acid with at least one organic base, water, and at least one water miscible organic solvent, wherein;
a. 0.1% to 40% by weight of at least one salt of ascorbic acid with an organic base;
b. 1% to 60% by weight of water; and,
c. 1% to 80% by weight of a water miscible organic solvent.

2. The water in claim 1 can come from other ingredients in the formula that may contain sufficient quantity of water.

3. The salts of ascorbic acid in claim 1 can be selected from a group consisting of, but not limited to, glucosamine ascorbate, arginine ascorbate, lysine ascorbate, glutathione ascorbate, nicotinamide ascorbate, niacin ascorbate, allantoin ascorbate, creatine ascorbate, creatinine ascorbate, chondroitin ascorbate, chitosan ascorbate, DNA Ascorbate, and carnosine ascorbate.

4. Additional skin, hair, or body beneficial ingredients may also be present in composition of claim 1.

5. The compositions in claim 1, wherein such compositions have the beneficial synergistic effect of, including but not limited to, reduction of signs of skin aging, enhancement of collagen synthesis of skin, enhancement of cellular regeneration of skin, connective tissue regeneration, acceleration of cell growth, rejuvenation of skin's healing ability, wound healing, scar reduction, age spot reduction, cellulite reduction, skin lightening, enhanced anti-oxidant protection, enhanced free-radical entrapment, enhanced anti-inflammatory effect, enhanced anesthetic effect, and enhanced photo-protection of skin.

6. The salts of ascorbic acid with an organic base in claim 1, wherein such salts are prepared in-situ.

7. Additional skin, hair, or body beneficial ingredients in claim 4, wherein such ingredient may be from 1.0% to 30% of a surfactant or mixture of surfactants.

8. Additional skin, hair, or body beneficial ingredients in claim 4, wherein such an ingredient may be from 0.1% to 20% of at least one rheology modifier.

9. The salts of ascorbic acid with an organic base in claim 6 that are prepared in situ by,
a. the reaction of ascorbic acid with an organic base in equimolar amounts in water, or,
b. the reaction of ascorbic acid with an organic base in equimolar amounts in a mixture of water and a water-miscible organic solvent.

10. The salts of ascorbic acid with an organic base in claim 6 that are prepared in situ by,
a. the reaction of a metal salt of ascorbic acid with an acid salt of an organic base in equimolar amounts in water, or,
b. the reaction of a metal salt of ascorbic acid with an acid salt of an organic base in equimolar amounts in a mixture of water and a water-miscible organic solvent.

11. The organic base or its derivatives in claim 9, wherein the amount of such base or its derivatives is sufficient to bind and neutralize ascorbic acid in the pH range of 4.0 to 7.5.

12. The metal salts of ascorbic acid in claim 10 can be its alkali or alkaline earth metal salts, including but not limited to, Sodium Ascorbate, Potassium Ascorbate, Calcium Ascorbate, Magnesium Ascorbate, Zinc Ascorbate, and Sodium Ascorbyl Phosphate.

13. The organic base in claim 9, wherein such organic base can be selected from skin, hair, and body beneficial ingredients including, but not limited to, amines, amino acids, amino acid esters, substituted purines, substituted pyrimidines, substituted nucleic acids, nucleotides, DNA, RNA, peptides, amino sugars, and their derivatives.

14. The organic base in claim 13, wherein such bases or their acid salt derivatives can be selected from, either alone or in combination, but not limited to, allantoin, glutathione, arginine, arginine esters, creatine, creatinine, carnosine, glucosamine, lysine, lysine esters, lysyl-arginine, lysyl-arginine esters, arginyl-lysine esters, chitosan, chondroitin, niacinamide, niacin, benzyl nicotinate, methyl nicotinate, yohimbine, quinine, reserpine, DNA, and inorganic acid salts of such bases.

15. The organic bases in claim 13, wherein such bases can be ethanolamine ingredients. The ethanolamine is selected from the group either alone or in combination, consisting of but not limited to, dimethylaminoethanol, monoaminoethanol, choline, serine, acetic acid esters of dimethylaminoethanol, acetic acid esters of monoaminoethanol, para-chlorophenylacetic acid esters of dimethylaminoethanol, para-chlorophenylacetic acid esters of monoaminoethanol, and mixtures thereof. These compounds are hypothesized to treat aging skin via a mechanism of neuromuscular stimulation.

16. The organic bases in claim 13, wherein such bases can be selected from topical anesthetics such as, but not limited to, benzocaine, dibucaine, dyclonine, lidocaine, pramoxine, tetracaine, ephedrine, epinephrine, and phenylephrine.

17. The derivatives of organic bases in claim 14, wherein such acid salt derivatives of organic base can be inorganic acid salts of said organic bases in claim 13, for example but not limited to, glucosamine sulfate, glucosamine hydrochloride, chondroitin sulfate, chondroitin hydrochloride, lysine hydrochloride, lysine sulfate, arginine hydrochloride, arginine sulfate, niacinamide hydrochloride, niacinamide sulfate, niacin hydrochloride, niacin sulfate, yohimbine hydrochloride, yohimbine sulfate, benzocaine hydrochloride, dibucaine hydrochloride, tetracaine hydrochloride, pramoxine hydrochloride, lidocaine hydrochloride, diclonine hydrochloride, ephedrine sulfate, epinephrine hydrochloride, and phenylephrine hydrochloride.
